(19) United States
(12) Patent Application Publication
(10) Pub. No.: US 2025/0099777 A1
(43) Pub. Date: Mar. 27, 2025

(54) LIGHT-THERAPEUTIC MOUTH GUARD

(71) Applicant: Hee Chan CHA, Seoul (KR)

(72) Inventor: Hee Chan CHA, Seoul (KR)

(21) Appl. No.: 18/725,791

(22) PCT Filed: Jan. 2, 2023
(86) PCT No.: PCT/KR2023/000010

§ 371 Date: Jun. 30, 2024

(30) Foreign Application Priority Data
Dec. 31, 2021 (KR) 10-2021-0194439

(51) Int. Cl. A61N 5/06

(52) U.S. Cl. CPC A61N 5/0603; A61N 5/0624; A61N 2005/0606; A61N 2005/0626; A61N 2005/0645; A61N 2005/0652; A61N 2005/0661; A61N 2005/0663

(57) ABSTRACT

The present invention relates to a light-therapeutic mouth guard that maximizes sterilization and anti-inflammatory effects on teeth and gingiva by increasing emission intensity through overlapping light-emitting elements. The invention includes a guard body, worn in the mouth, accommodating teeth and gingiva with an accommodation groove. The groove consists of a masticatory surface-facing part that corresponds to the teeth row, and inner and outer wall parts facing the teeth and gingiva from both sides. A side light-emitting element is provided on the inner and/or outer wall part of the guard body to emit light at the side surfaces of the gingiva and teeth. The side light-emitting element is arranged in multiple rows to overlap the emitted light, thereby increasing emission intensity.

### TECHNICAL FIELD

The present invention relates, in general, to a mouthguard for light therapy and, more particularly, to a mouthguard for light therapy that maximizes the sterilization and anti-inflammatory effects on teeth and gums by increasing the intensity of irradiation through overlapping illumination of light-emitting components.

### BACKGROUND ART

Generally, teeth serve an important function in vertebrates' digestive systems by finely chewing and grinding food, while the gums are part of the oral mucosa covering the teeth and the alveolar processes of the mandible.

A prominent method for managing such teeth and gums is brushing, but in Korean Patent Application No. 2015-0002011 (published on Jan. 7, 2016), titled “Mouthpiece,” technology has been disclosed wherein LEDs are installed within the main body of the mouthpiece to irradiate red and blue light towards the teeth, thereby obtaining sterilization and anti-inflammatory effects.

However, in the “Mouthpiece” of Korean Patent Application No. 2015-0002011, the light emitted from the LEDs was configured to be focused on the occlusal surface of the teeth. Therefore, while some sterilization effects on the teeth could be expected, there were limitations in expecting sterilization or anti-inflammatory effects on the gums.

Furthermore, due to the limited intensity of the LED light irradiation, it was not easy to achieve a sufficient level of sterilization and anti-inflammatory effects.

### DISCLOSURE

### Technical Problem

Therefore, the present invention is proposed to address the aforementioned conventional problems. The objective of the present invention is to provide a mouthguard for light therapy that maximizes sterilization and anti-inflammatory effects on teeth and gums by increasing the irradiation intensity through overlapping illumination of light-emitting components.

### Technical Solution

The mouthguard for light therapy, based on the technical idea of the present invention, is designed to achieve the aforementioned objectives. It includes a guard body that can be worn in the oral cavity and accommodates teeth and gums. This guard body consists of an occlusal surface facing portion with a curvature corresponding to that of the teeth, facing the occlusal surface of the teeth, and inner and outer wall portions facing the inner and outer sides of the teeth and gums, respectively. Additionally, the mouthguard features side light-emitting components installed on at least one of the inner and outer wall portions of the guard body. These components are capable of irradiating light towards the sides of the gums and teeth. Notably, the technical configuration of the side light-emitting components involves using multiple LEDs to allow the irradiated light to overlap, thereby increasing the irradiation intensity.

Here, the side light-emitting components comprise a plurality of first light-emitting elements generating light at a first wavelength for irradiation and formed into a first light-emitting element array, and a plurality of second light-emitting elements generating light at a second wavelength for irradiation and formed into a second light-emitting element array.

Additionally, the first light-emitting element array and the second light-emitting element array may be arranged alternately with each other.

Furthermore, the first light-emitting element array and the second light-emitting element array may form horizontal rows along the longitudinal direction of the guard body.

Furthermore, the horizontal rows of the first light-emitting elements and the second light-emitting elements alternate from the gums towards the teeth. Specifically, the light emitted from the horizontal rows of the first light-emitting elements corresponding to the first column and the third column overlaps on the gums, while the light emitted from the horizontal rows of the second light-emitting elements corresponding to the second column and the fourth column overlaps on the gingival margin.

Additionally, the horizontal rows of the first light-emitting elements and the second light-emitting elements may be configured to form a waveform corresponding to the gingival margin.

Furthermore, the first light-emitting elements and the second light-emitting elements may form vertical columns along the vertical direction of the guard body.

Additionally, the vertical columns of the second light-emitting elements are positioned between and adjacent to the vertical columns of the first light-emitting elements on both sides. This arrangement allows the light emitted from the vertical columns of the first light-emitting elements, corresponding to the gingiva and teeth, to overlap. Similarly, the light emitted from the vertical columns of the second light-emitting elements, positioned between and adjacent to the vertical columns of the first light-emitting elements, also overlaps on the gingiva and teeth.

Additionally, the first light-emitting elements and the second light-emitting elements may form diagonal rows with respect to the gingival margin on the guard body. Furthermore, the first light-emitting elements and the second light-emitting elements may form diagonal rows with respect to the gingival margin on the guard body. Additionally, the diagonal rows of the second light-emitting elements are positioned between and adjacent to the diagonal rows of the first light-emitting elements on both sides. This configuration ensures that the light emitted from the diagonal rows of the first light-emitting elements, corresponding to the gingiva and teeth, overlaps. Similarly, the light emitted from the diagonal rows of the second light-emitting elements, positioned between and adjacent to the diagonal rows of the first light-emitting elements, also overlaps on the gingiva and teeth.

Additionally, the first light-emitting elements and the second light-emitting elements may each be equipped with RGB light-emitting elements capable of selectively emitting light at multiple wavelengths within a wavelength range.

Furthermore, the RGB light-emitting elements may form horizontal rows along the longitudinal direction of the guard body. These rows of RGB elements emitting light at the first and second wavelengths are arranged in a repeating pattern with a certain pattern of alternating between them.

Furthermore, the RGB light-emitting elements allow the light emitted from the horizontal rows of RGB elements corresponding to the first column and the third column to overlap on the gums, while the light emitted from the horizontal rows of RGB elements corresponding to the second column and the fourth column overlaps on the gingival margin.

Additionally, the RGB light-emitting elements may form vertical columns along the vertical direction of the guard body, extending from the gums to the teeth. These columns of RGB elements emitting light at the first and second wavelengths are arranged in a repeating pattern with a certain pattern of alternating between them.

Furthermore, the light emitted at the first wavelength is red light, while the light emitted at the second wavelength is blue light.

Additionally, at least some of the first light-emitting elements and the second light-emitting elements may generate far-UVC radiation for irradiation.

Furthermore, at least some of the first light-emitting elements and the second light-emitting elements may generate ultraviolet-B (UVB) radiation for irradiation.

Furthermore, the guard body may include additional occlusal surface light-emitting elements installed on the occlusal surface facing portion of the guard body, enabling irradiation towards the occlusal surface of the teeth.

Furthermore, the occlusal surface light-emitting elements may be equipped with blue light-emitting elements capable of emitting blue light for the purpose of sterilizing the teeth.

Furthermore, the blue light-emitting elements may form multiple horizontal rows along the longitudinal direction of the guard body. This configuration allows the blue light emitted from the blue light-emitting elements to overlap on the occlusal surface of the teeth, thereby increasing the intensity of the blue light irradiation.

Furthermore, the occlusal surface light-emitting elements may be equipped with RGB light-emitting elements capable of selectively emitting light at multiple wavelengths within a wavelength range.

Furthermore, at least some of the light-emitting components on the occlusal surface are capable of emitting far-UVC radiation for irradiation.

Furthermore, at least some of the occlusal surface light-emitting elements may generate far-UVC radiation for irradiation.

Furthermore, the guard body may include a plurality of ultrasonic transducers embedded along the longitudinal direction of the guard body, enabling induction of ultrasonic vibrations on the teeth to facilitate cleaning.

Furthermore, the guard body may include a vibration transmitting member that protrudes from the guard body into the accommodation cavity when connected to the ultrasonic transducers, allowing the vibration transmitting member to come into contact with the teeth and induce ultrasonic vibrations on the teeth.

Furthermore, the ultrasonic transducers may be arranged along the longitudinal direction of the guard body to individually address each tooth, allowing for separate operation. This enables the application of ultrasonic vibrations to each tooth with time intervals according to a programmed schedule or focusing intense ultrasonic vibrations on specific teeth as needed.

Furthermore, two mouthguards can be paired together to allow simultaneous wearing on the upper and lower jaws. The side light-emitting components of the paired mouthguards worn on the upper and lower jaws together can be integrated and controlled through a communication module.

Furthermore, the two mouthguards can be paired together to allow simultaneous wearing on the upper and lower jaws. Each guard body of the paired mouthguards is equipped with fastening means to secure them to each other when worn on the upper and lower jaws simultaneously.

Furthermore, the accommodation cavity of the guard body consists of an upper accommodation cavity opened upward and a lower accommodation cavity opened downward, allowing for simultaneous wearing on the upper and lower jaws. Each of the upper and lower accommodation cavities is equipped with the side light-emitting components.

Furthermore, it may include a controller to control the operation of the side light-emitting components and a communication module for external communication.

Meanwhile, the light therapy system according to the present invention includes the aforementioned light therapy mouthguard and a terminal device equipped with a dedicated app installed for controlling the side light-emitting components via the controller.

Here, the first light-emitting elements and the second light-emitting elements are each equipped with RGB light-emitting elements capable of selectively emitting light at multiple wavelengths within a wavelength range. The dedicated app includes a color palette where the user can choose one color from multiple options. When the user selects a color from the color palette in the dedicated app, the controller controls the RGB light-emitting elements to emit light of the selected color for irradiation.

Furthermore, the display of the terminal device running the dedicated app shows a dental arch image, and the user can select individual tooth images within the dental arch image by clicking on them to choose which of the side light-emitting components should emit light for irradiation.

Furthermore, it includes additional occlusal surface light-emitting elements installed on the occlusal surface facing portion of the guard body to emit light towards the occlusal surface of the teeth. The dedicated app enables control of these occlusal surface light-emitting elements via the controller, allowing the user to select which of the side light-emitting components and occlusal surface light-emitting elements should emit light for irradiation by clicking on individual tooth images displayed on the terminal device's display.

Furthermore, the occlusal surface light-emitting elements are equipped with RGB light-emitting elements capable of selectively emitting light at multiple wavelengths within a wavelength range. The dedicated app includes a color palette where the user can choose one color from multiple options. When the user selects a color from the color palette in the dedicated app, the controller controls the RGB light-emitting elements to emit light of the selected color for irradiation.

### Advantageous Effects

The mouth guard for light therapy according to the present invention can maximize the sterilizing and anti-inflammatory effects on teeth and gums by increasing the intensity of irradiation through overlapping irradiation of light-emitting elements made up of a plurality of rows.

In addition, the present invention cleans and removes causative substances such as fine food debris and plaque that cause bacterial growth and inflammation in teeth and gums by using an ultrasonic vibrator installed in response to tooth row along with overlapping irradiation of light-emitting elements. This can further enhance the sterilizing and anti-inflammatory effects on teeth and gums.

### DESCRIPTION OF DRAWINGS

FIG. 1 is a perspective view of a mouth guard for light therapy according to an embodiment of the present invention.

FIG. 2 is a modified development diagram for explaining the LED arrangement of the mouth guard for light therapy according to an embodiment of the present invention.

FIG. 3 is a cross-sectional view of a mouth guard for light therapy according to an embodiment of the present invention.

FIG. 4a is a state in which a pair of mouth guards for light therapy according to an embodiment of the present invention are worn on the upper and lower jaw.

FIG. 4b is a state in which a pair of mouth guards for light therapy according to an embodiment of the present invention are fastened by a fastening member and worn on the upper and lower jaw.

FIG. 5 is a cross-sectional view of a mouth guard for light therapy according to a first modified embodiment of the present invention;

FIG. 6 is a perspective view of a mouth guard for light therapy according to a second modified embodiment of the present invention; and

FIG. 7 is a cross-sectional view of a mouth guard for light therapy according to a second modified embodiment of the present invention.

FIG. 8 is a modified development diagram for explaining the LED arrangement of the mouth guard for light therapy according to the second modified embodiment.

FIG. 8 is a perspective view of the mouth guard for light therapy according to the third modified embodiment of the present invention.

FIG. 9 is a third modified version of the present invention. A modified development diagram for explaining the LED arrangement of the mouth guard for light therapy according to the embodiment.

FIG. 10 is a perspective view of the mouth guard for light therapy according to a fourth modified embodiment of the present invention.

FIG. 11 is a fourth modified embodiment of the present invention.

FIG. 12 is a modified development diagram for explaining the LED arrangement state of the mouth guard for light therapy according to an embodiment of the present invention.

FIG. 13 is a configuration diagram of a light therapy system using a mouth guard for light therapy according to an embodiment of the present invention. Front view of a terminal with a dedicated app installed in a light therapy system using a light therapy mouth guard

A mouth guard for light therapy according to embodiments of the present invention will be described in detail with reference to the attached drawings. Since the present invention can be subject to various changes and can have various forms, specific embodiments will be illustrated in the drawings and described in detail in the text. However, this is not intended to limit the present invention to a specific disclosed form, and should be understood to include all changes, equivalents, and substitutes included in the spirit and technical scope of the present invention. While describing each drawing, similar reference numerals are used for similar components. In the attached drawings, the dimensions of the structures are enlarged from the actual size for clarity of the present invention, or reduced from the actual size to understand the schematic configuration.

Additionally, terms such as first and second may be used to describe various components, but the components should not be limited by the terms. The above terms are used only for the purpose of distinguishing one component from another. For example, a first component may be named a second component, and similarly, the second component may also be named a first component without departing from the scope of the present invention. Meanwhile, unless otherwise defined, all terms used herein, including technical or scientific terms, have the same meaning as generally understood by those skilled in the art to which the present invention pertains. Terms such as those defined in commonly used dictionaries should be interpreted as having a meaning consistent with the meaning in the context of the related technology, and unless clearly defined in the present application, should not be interpreted as having an ideal or excessively formal meaning.

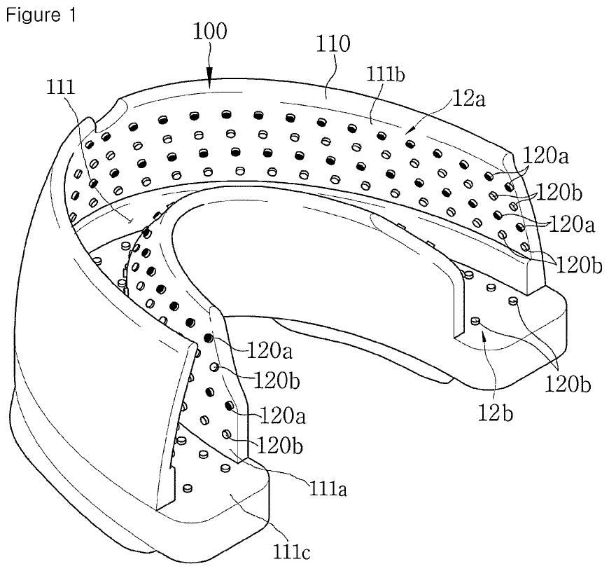

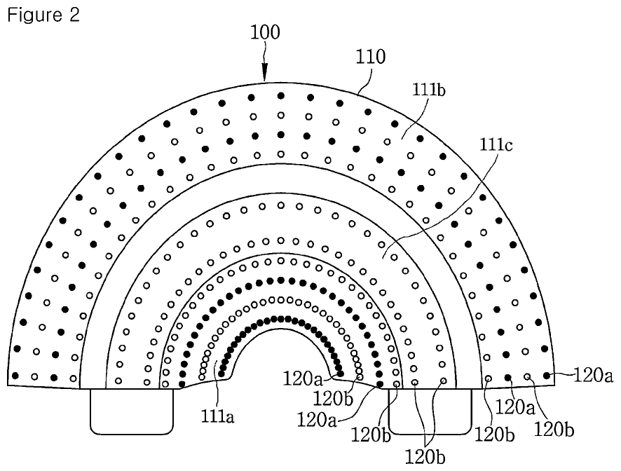

FIG. 1 is a perspective view of a mouth guard for light therapy according to an embodiment of the present invention, and FIG. 2 is a modified development view for explaining the LED arrangement state of the mouth guard for light therapy according to an embodiment of the present invention. 3 is a cross-sectional view of a mouth guard for light therapy according to an embodiment of the present invention.

As shown, the mouth guard 100 for light therapy according to an embodiment of the present invention includes a guard body 110 in the form of a mouthpiece that can be worn in the mouth, and an inner wall portion of the guard body 110 (111a), a side light-emitting device 12a installed on the outer wall portion 111b, a chewing surface light-emitting device 12b installed on the chewing surface of the guard body 110, and an ultrasonic vibrator 130 as the main components. do. The present invention can increase the irradiation intensity in a unique way in which the light generated from the side light-emitting device 12a and the chewing surface light-emitting device 12b, which consists of a plurality of rows, is irradiated to the gums (S1) and teeth (T1) in an overlapping form. It is designed to maximize the sterilizing and anti-inflammatory effects on teeth (T1) and gums (S1).

Hereinafter, the mouth guard 100 for light therapy according to an embodiment of the present invention will be described in detail, focusing on each of the above components.

The guard body 110 has a mouthpiece shape as shown in FIG. 1 and is designed to be easily worn in the mouth. The guard body 110 has a chewing surface facing portion 111c that has a curvature corresponding to the tooth row and faces the chewing surface of the tooth T1, and faces the tooth T1 and the gum S1 from the inside and outside, respectively. It has a receiving groove 111 consisting of an inner wall portion 111a and an outer wall portion 111b. The guard body 110 can be simply worn in the oral cavity by accommodating and surrounding the teeth (T1) and gums (S1) through the receiving grooves (111). Here, the guard body 110 is preferably made of silicone or a similar material with flexibility and elasticity and is manufactured so that it can be comfortably worn on the upper and lower jaw with some extra space.

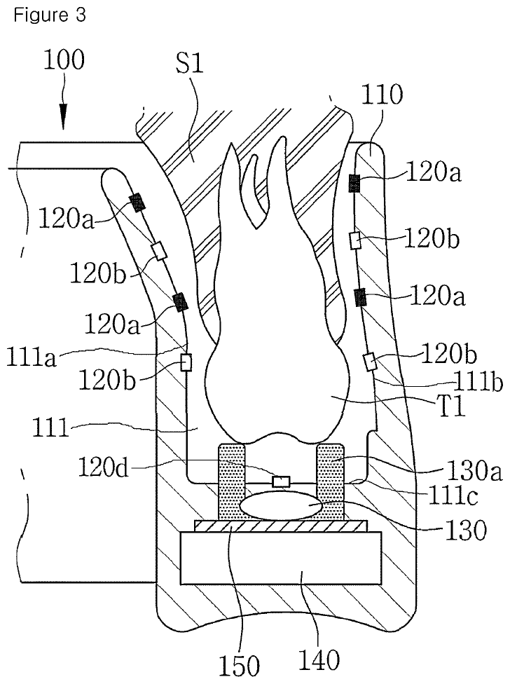

The side light emitting element (12a) emits red light as the first wavelength light toward the side of the tooth (T1) and gum (S1) from the inner wall portion (111a) and the outer wall portion (111b) of the guard body 110. and is installed to irradiate blue light as the second wavelength light. Red light is relatively effective for the formation of alveolar bone and anti-inflammatory at the root of the tooth (T1), while blue light is relatively more effective for sterilization. In an embodiment of the present invention, as shown in FIGS. 1 to 3, a red LED 120a as a first light-emitting device that irradiates light in the first wavelength band is installed to form a horizontal row along the longitudinal direction of the guard body 110. As a second light-emitting device that irradiates light in the second wavelength band, a blue LED (120b) is installed to form a horizontal row along the longitudinal direction of the guard body (110).

It can be noted that the horizontal rows of the red LEDs (120a) and the horizontal rows of the blue LEDs (120b) are alternately arranged from the gums (S1) toward the teeth (T1). Red light generated from the horizontal row of red LEDs 120a corresponding to the first row and the horizontal row of red LEDs 120a corresponding to the third row are overlapped and irradiated to the gum S1. For this purpose, the horizontal row of red LEDs 120a corresponding to the first row and the horizontal row of red LEDs 120a corresponding to the third row are positioned to face the gum S1 although there is a step between them. As a result, the red light generated from the horizontal rows of the red LEDs 120a corresponding to the first row and the horizontal rows of the red LEDs 120a corresponding to the third row are overlapped and irradiated to the gums S1 to increase the irradiation intensity, forming alveolar bone, It can achieve higher effectiveness for anti-inflammation at the root of the tooth (T1).

Meanwhile, the blue light generated from the horizontal row of blue LEDs (120b) corresponding to the second row and the horizontal row of blue LEDs (120b) corresponding to the fourth row are overlapped and irradiated to the gingival border. For this purpose, the horizontal row of blue LEDs (120b) corresponding to the second row and the horizontal row of blue LEDs (120b) corresponding to the fourth row are installed so as to be located at the upper and lower sides with the gingival border between them. As a result, the blue light generated from the horizontal rows of the blue LEDs 120b corresponding to the second row and the horizontal rows of the blue LEDs 120b corresponding to the fourth row are overlapped and irradiated to the gingival interface, thereby increasing the intensity of irradiation. In particular, the proliferation of bacteria is concentrated. It is possible to achieve a higher sterilization effect on the gingival interface, which is prone to damage.

The red LED (120a) uses a wavelength range of 630 to 690 nm, and the blue LED (120b) uses a wavelength range of 400 to 450 nm.

A plurality of the chewing surface light emitting devices 12b are installed on the chewing surface facing portion 111c of the guard main body 110 to irradiate light toward the chewing surface of the tooth T1. The chewing surface light emitting device 12b is preferably provided with a plurality of blue LEDs 120b arranged in a row to generate blue light for sterilization of the teeth T1. In an embodiment of the present invention, the blue LEDs 120b are provided in a form that forms a plurality of horizontal rows parallel to each other along the longitudinal direction of the guard body 110. According to the configuration in which the blue LEDs 120b are provided in a plurality of horizontal rows on the chewing surface facing portion 111c of the guard main body 110, the blue lights are irradiated to the chewing surface of the tooth T1 in an overlapping state, thereby irradiating the blue light. Strength can be increased. As a result, it is possible to maximize the sterilization effect of blue light on the chewing surface of the tooth T1.

In the case of the blue LED (120b), a wavelength range of 400 to 450 nm is used.

The LEDs constituting the side light emitting device 12a and the bottom light emitting device 12b are each configured to emit independent individual light. As a result, various types of light emission are possible, such as the individual LEDs in the horizontal row of the red LEDs 120a and the horizontal row of the blue LEDs 120b emitting light sequentially with a time difference or emitting light intensively in some parts, according to the program.

Meanwhile, although not shown in the drawing, the horizontal rows of the red LEDs (120a) and the horizontal rows of the blue LEDs (120b) can be modified to draw a waveform corresponding to the gingival boundary surface. In this way, when the horizontal rows of the red LED (120a) and the horizontal rows of the blue LED (120b) are formed to draw a wave shape corresponding to the gingival interface, light irradiation can be performed with a higher concentration on the gingival interface, which is a site with a high possibility of bacterial growth. There is an advantage to having it.

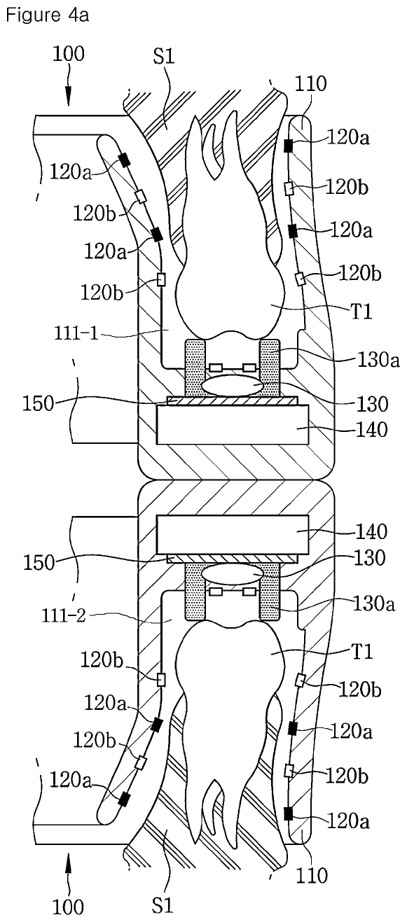

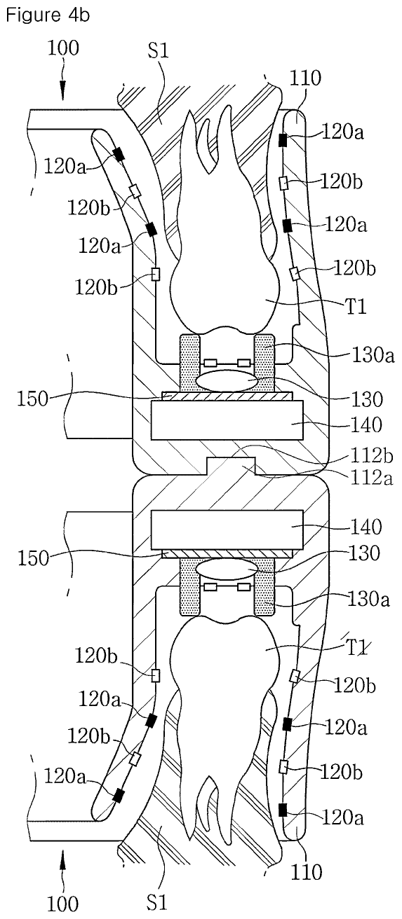

A light therapy mouth guard (100) as described can be worn in pairs, as illustrated in FIG. 4a, on both the upper and lower jaws simultaneously. In this scenario, it is preferable for the pair of mouth guards (100) to be interconnected and integrally controlled through embedded communication modules. To ensure comfortable wearing of the light therapy mouth guard (100) on both the upper and lower jaws, it is advisable for each guard body (110) of the pair, as depicted in FIG. 4b, to be equipped with fastening mechanisms that securely join them together when worn on the upper and lower jaws. These fastening mechanisms, as shown in FIG. 4b, can be simply designed as interlocking pegs (112a) and sockets (112b) that mate with each other, and other forms with similar functionality are also applicable without limitation.

The ultrasonic transducers (130) are embedded in the guard body (110), arranged in multiple units along the length of the guard body (110), allowing them to induce ultrasonic vibrations on the teeth (T1) for cleaning purposes. By enabling cleaning through ultrasonic vibrations, it becomes possible to remove tiny food debris and plaque, which are the causes of bacterial growth and inflammation. This contributes significantly to the effective management of both the teeth (T1) and the gums (S1), in addition to the germicidal and anti-inflammatory effects of light irradiation. It is desirable for the ultrasonic transducers (130) to be installed along the length of the guard body (110), allowing each transducer to be individually operated and arranged to cater to each tooth (T1) separately. This enables various operations as needed, such as applying ultrasonic vibrations with time intervals for each tooth (T1) or selectively targeting specific teeth (T1) with intensified ultrasonic vibrations.

It is desirable to include a vibration transmitting member (130a) to effectively deliver the ultrasonic vibrations of the ultrasonic transducers (130) to the teeth (T1). The vibration transmitting member (130a) is installed such that its distal end protrudes into the receptacle (111) of the guard body (110) while the proximal end is connected to the ultrasonic transducer (130), allowing it to make contact with the teeth (T1) when attached to the guard body (110).

In one embodiment of the present invention, the light therapy mouthguard, attached to a substrate (150), incorporates a compact rechargeable battery (140) for ease of portable use outdoors without the need for separate power connections. Additionally, it may include a controller that individually regulates the separate LEDs within the side light emitter (12a) and the occlusal surface light emitter (12b), along with a communication module for external communication. This allows for the establishment of a light therapy system that can be conveniently and effectively used with external devices such as smartphones, enabling more user-friendly operation. Further details regarding the light therapy system will be elaborated upon in subsequent descriptions.

### MODE FOR INVENTION

### First Variant Embodiment

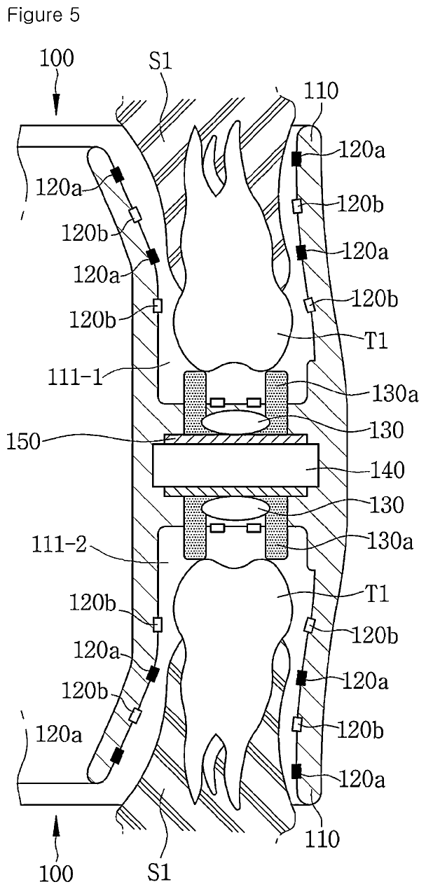

FIG. 5 is a cross-sectional view of a light therapy mouth guard according to a first modified embodiment of the present invention.

As illustrated, according to the first modified embodiment of the present invention, the receptacle of the mouth guard (110) is comprised of an upper receptacle (111-1) open towards the upper side and a lower receptacle (111-2) open towards the lower side, enabling it to be worn on both the upper and lower jaws. Notably, each of the upper receptacle (111-1) and lower receptacle (111-2) is equipped with side-emitting light-emitting diodes (12a) and occlusal surface light-emitting diodes (12b).

In addition to the installation of side-emitting light-emitting diodes (12a) and occlusal surface light-emitting diodes (12b) on the upper receptacle (111-1) and lower receptacle (111-2) of the guard body (110) respectively, ultrasonic vibrators (130) and vibration transmitting members (130a) are also installed on the upper and lower parts. However, it is permissible to install one charging small-sized battery (140), controller, and communication module each.

According to this first variant embodiment, one of the advantages of the present invention is that with just one mouse guard, it is possible to simultaneously manage the gums (S1) and teeth (T1) in both the upper and lower jaws.

However, for other components not described in this first variant embodiment, they are substantially similar to those in the original embodiment, so detailed explanation is omitted.

### Second Variant Embodiment

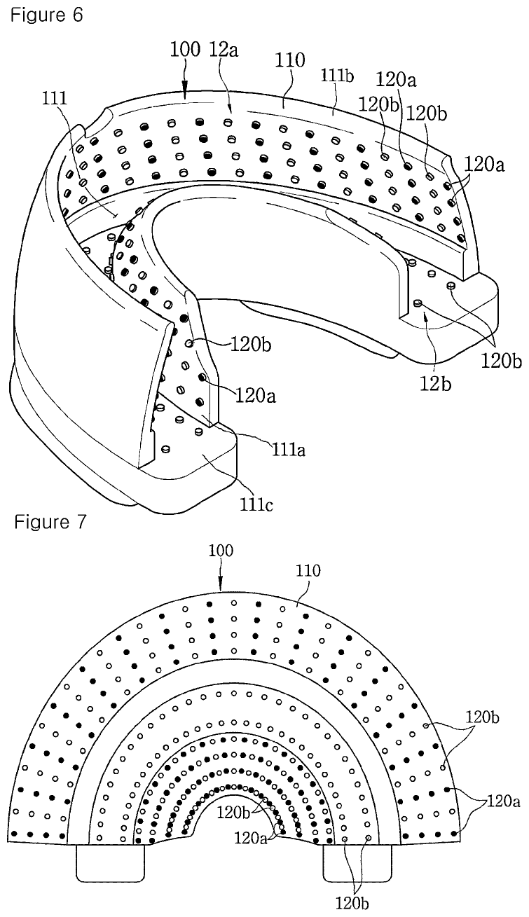

FIG. 6 depicts a lateral view of the light therapy mouse guard according to the second variant embodiment of the present invention, while FIG. 7 illustrates a schematic diagram showing the arrangement of LEDs for the light therapy mouse guard according to the second variant embodiment of the present invention.

As illustrated, the second variant embodiment of the present invention comprises red LEDs (120a) and blue LEDs (120b) forming vertical rows along the vertical direction of the guard body (110), creating lateral illumination. These red LEDs (120a) and blue LEDs (120b) are alternately arranged along the longitudinal direction of the guard body (110).

Here, considering a single red LED (120a) as a reference, it extends from the gum (S1) to the tooth (T1), and the vertical rows of blue LEDs (120b) are similarly configured. Consequently, the red light emitted from the vertical rows of red LEDs (120a), which are adjacent to each other on either side of the vertical rows of blue LEDs (120b), overlaps and illuminates both the gum (S1) and the tooth (T1). Likewise, the blue light emitted from the vertical rows of blue LEDs (120b), which are adjacent to each other on either side of the vertical rows of red LEDs (120a), overlaps and illuminates both the gum (S1) and the tooth (T1).

In this manner, the second variation example allows for almost uniform overlapping irradiation of both red and blue lights on the tooth (T1), gum (S1), and gumline, contrasting with the previous embodiment.

Here, it is desirable for the red LEDs (120a) and blue LEDs (120b) to be individually controllable, similar to the previous embodiment. With this configuration, various operations become possible, including simultaneous illumination of the vertical columns of red LEDs (120a) and blue LEDs (120b), illumination of only one of the vertical columns of red LEDs (120a) or blue LEDs (120b), or selective illumination of some LEDs within the vertical columns of red LEDs (120a) and blue LEDs (120b).

However, since other components not described in the second embodiment of the present invention are largely similar to those of the previous embodiment, detailed explanation is omitted.

Meanwhile, although not explicitly depicted in the drawings, the second embodiment can be applied such that the side-emitting light-emitting diodes (LEDs) (12a) are equipped with red LEDs (120a) and blue LEDs (120b) forming diagonal rows along the gingival boundary on the guard body (110). The diagonal rows of red LEDs (120a) and blue LEDs (120b) enable nearly uniform overlapping of red and blue lights on the teeth (T1), gums (S1), and gingival boundary, much like in the second embodiment.

Here, it is desirable for the red LEDs (120a) and blue LEDs (120b) to be individually controllable for illumination. This configuration allows for various operations, including simultaneous illumination of the diagonal rows of red LEDs (120a) and blue LEDs (120b), as well as selective illumination of either the diagonal rows of red LEDs (120a) or blue LEDs (120b), or even partial illumination of some LEDs within the diagonal rows of red LEDs (120a) and blue LEDs (120b).

### Third Variation Embodiment

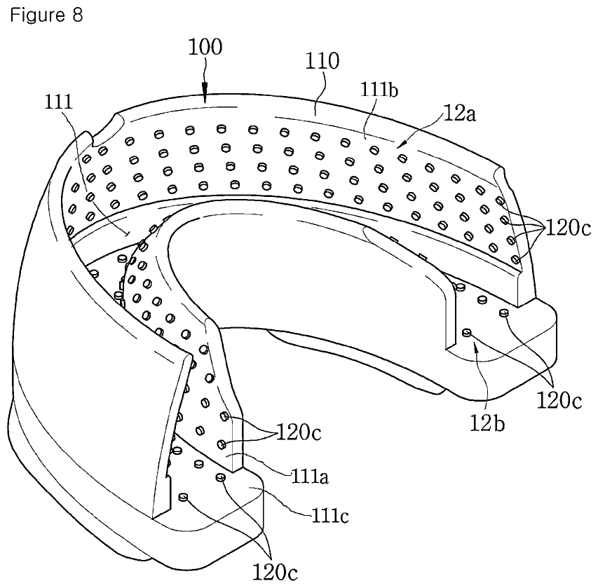

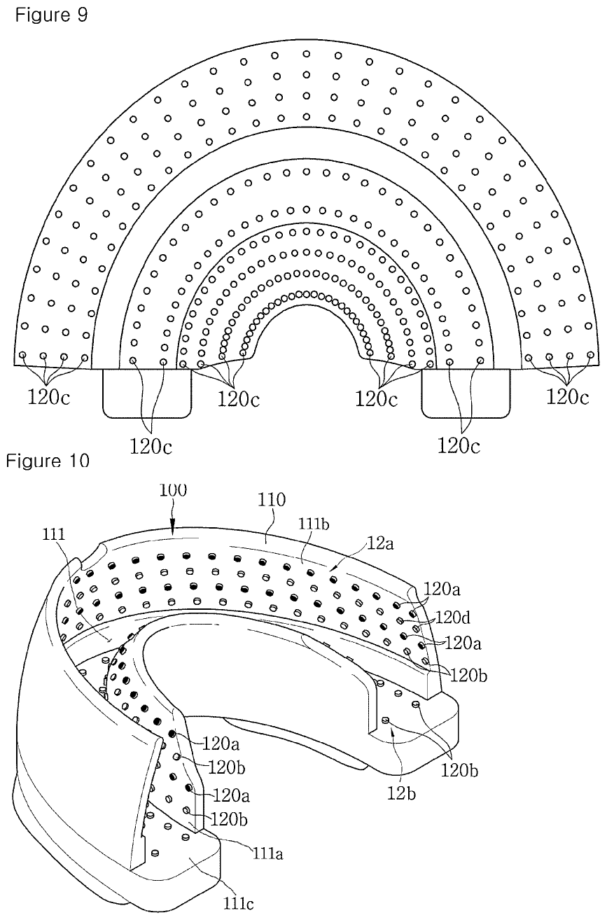

FIG. 8 depicts a perspective view of the light therapy mouth guard according to the third variation embodiment of the present invention, while FIG. 9 illustrates a layout diagram of the LEDs in the light therapy mouth guard according to the third variation embodiment of the present invention.

As illustrated, the third variation embodiment of the present invention is characterized by the side-emitting LEDs (12a) and the occlusal surface-emitting LEDs (12b) being equipped with RGB LEDs (120c) capable of selectively emitting light at one of multiple wavelengths.

For the side-emitting LEDs (12a), the horizontal rows of RGB LEDs (120c) corresponding to the first and third rows can be positioned to face the gingiva (S1), while the horizontal rows corresponding to the second and fourth rows can be installed above and below the gingival margin, respectively.

This allows the side-emitting LEDs (12a) to emit red light from the horizontal rows of RGB LEDs (120c) corresponding to the first and third rows, overlapping onto the gingiva (S1), while emitting blue light from the horizontal rows corresponding to the second and fourth rows, overlapping onto the gingival margin. Conversely, it's possible to emit blue light from the horizontal rows of RGB LEDs (120c) corresponding to the first and third rows, overlapping onto the gingiva (S1), and emit red light from the horizontal rows corresponding to the second and fourth rows, overlapping onto the gingival margin.

Furthermore, according to the third variant embodiment, by arranging the RGB LEDs (120c) along the vertical direction of the guard body (110) from the gingiva (S1) to the teeth (T1), it's possible to create vertical columns of red light and blue light alternately along the length of the guard body (110). This arrangement allows the red light emitted from the adjacent vertical columns of red light, positioned on either side of the blue light columns, to overlap onto both the gingiva (S1) and the teeth (T1), and similarly, the blue light emitted from the adjacent vertical columns of blue light, positioned on either side of the red light columns, to overlap onto both the gingiva (S1) and the teeth (T1). This corresponds to the operation of the RGB LEDs (120c) as described in the second variant embodiment.

Thus, in the third variant embodiment, by utilizing RGB LEDs (120c) to selectively emit red and blue light for irradiation, it's possible to achieve not only forms similar to the previous embodiment or the second variant embodiment, but also various operations such as alternating the arrangement of columns emitting red light and blue light diagonally, or alternating the arrangement of rows emitting red light and rows emitting blue light. Additionally, there is an advantage in being able to create various illumination patterns that may be necessary for the management of teeth (T1) and gingiva (S1), such as sparkling effects or wavelike effects, similar to an LED display board.

However, as for other components not described in the third variant embodiment of the present invention, they are similar to those in the previous embodiment, and therefore, detailed explanations are omitted.

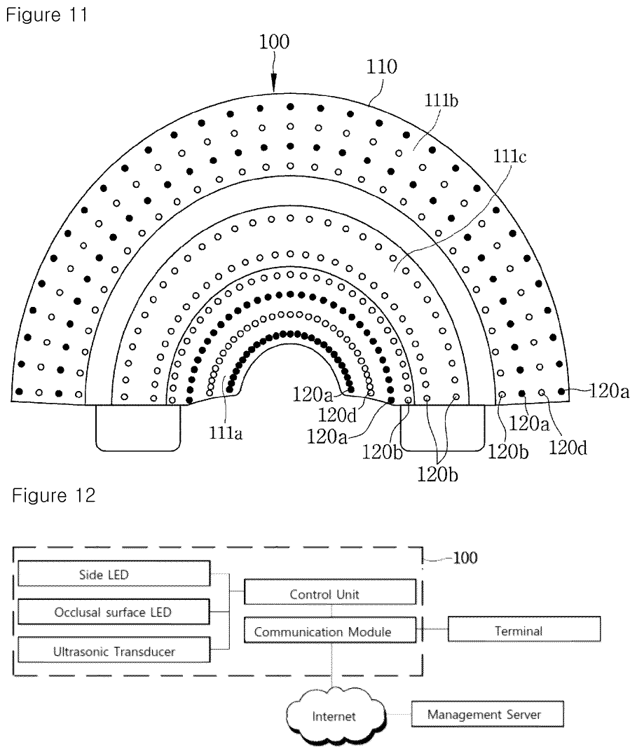

FIG. 10 depicts a perspective view of a mouse guard for light therapy according to the fourth variant embodiment of the present invention, while FIG. 11 illustrates the arrangement of LEDs for the mouse guard for light therapy according to the fourth variant embodiment of the present invention.

As depicted, the fourth variant embodiment of the present invention features at least some of the side-emitting light sources (12a) and the occlusal surface-emitting light sources (12b) equipped with UVC LEDs (120d) capable of emitting far-UVC radiation at a wavelength of 222 nm for illumination. However, it is noted that in the diagram, a UVC LED (120d) is shown installed in the horizontal row of the second column of the side-emitting light sources (12a).

It is noteworthy that the atomic ultraviolet radiation emitted by the UVC LEDs (120d) at a wavelength of 222 nm overcomes the safety limitations that have hindered its application in the human body thus far, enabling safe and effective sterilization.

However, detailed explanations of other components not described in the fourth exemplary embodiment of the present invention are omitted, as they remain substantially similar to those of the pre-modified embodiments.

Additionally, as another variant of the aforementioned fourth exemplary embodiment of the present invention, at least some of the side-emitting light-emitting diodes (LEDs) (12a) and the occlusal surface light-emitting diodes (12b) may be equipped with UV-B LEDs to emit and irradiate ultraviolet-B radiation.

The ultraviolet-B (UV-B) radiation emitted by UV-B LEDs refers to ultraviolet light in the wavelength range of 280 to 320 nanometers (nm). It is known to break down 7-dehydrocholesterol (7-DHC), which is commonly present in the skin tissue, into vitamin D3, thereby aiding in the smooth transportation of calcium (Ca) to strengthen bones. When exposed to such UV-B radiation, teeth (T1) and gum tissue (S1) are activated, promoting immune function and strengthening periodontal health, thus benefiting the overall health of teeth and gums.

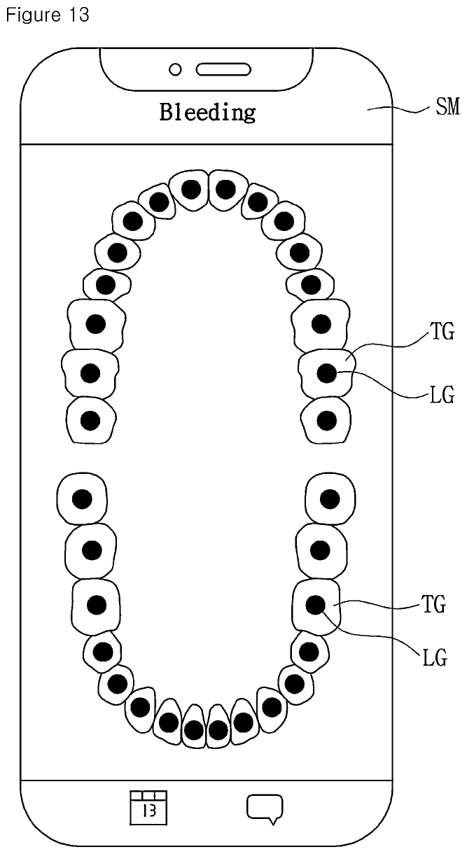

FIG. 12 illustrates a schematic diagram of a light therapy system utilizing a light therapy mouse guard according to an embodiment of the present invention, and FIG. 13 depicts a front view of a terminal device with a dedicated app installed, utilized in the light therapy system employing the light therapy mouse guard according to an embodiment of the present invention.

As depicted, the light therapy system according to an embodiment of the present invention consists of a light therapy mouse guard (100) equipped with a controller and communication module, along with a terminal device (SM) with a dedicated app installed, and a management server.

The aforementioned controller is equipped to individually control the side illuminators (12a) and the lower illuminators (12b), as well as the ultrasonic vibrator (130).

The aforementioned communication module is integrated into the mouse guard for communication with external devices. This communication module may utilize methods such as Direct Wi-Fi to directly transmit and receive information with the terminal (SM), and may also utilize other methods such as short-range communication networks and long-distance communication networks.

The terminal (SM) equipped with a display is a compact device, primarily referring to widely available smartphones, but may also include specially designed dedicated terminals. This terminal (SM) is installed with a dedicated app that communicates with the control unit of the mouse guard (100) through the communication module, enabling individual control of the side illuminators (12a), the bottom illuminators (12b), and the ultrasonic vibrator (130).

When the dedicated app is installed and launched on the terminal (SM), as shown in FIG. 13, a tooth image is displayed on the display, and individual LEDs belonging to the side illuminators (12a) and the bottom illuminators (12b) can be selected to illuminate by clicking on the touch area (LG) of individual tooth (T1) images (TG) within the tooth image. It is possible to select one or more individual LEDs belonging to the side illuminators (12a) and the bottom illuminators (12b) to illuminate. Furthermore, through the dedicated app, it is possible to configure settings such as the duration of illumination and the mode of illumination.

Furthermore, if the side illuminators (12a) and bottom illuminators (12b) are equipped with RGB illuminators capable of selectively generating light in one of multiple wavelengths, the dedicated app is designed to implement a color palette (or a color spectrum allowing for selection of a total natural color) on the display, allowing the user to choose any one color from the palette. Thus, when a user selects a color from the color palette displayed on the screen, the controller controls the RGB illuminators to emit light of the selected color for illumination. With this configuration, users can experiment with various colors of light and experience their effects firsthand. Moreover, these individual user experiences can be transmitted to the management server through the dedicated app, accumulating experiential data that may establish the effects of light exposure in specific, previously unknown colors.

Additionally, for the ultrasound vibrator (130), individual operation and operation time settings can also be enabled through the dedicated app, allowing focused management on specific teeth (T1) only.

While exemplary embodiments of the present invention have been described above, it should be noted that the invention is not limited to these embodiments and can be modified and varied using various changes and equivalents. It is evident that the same principles can be applied to appropriately modify the embodiments described above. Therefore, the scope of the present invention is not limited by the description provided above but by the claims below.
110: Guard body 111: Receiving groove
12a: Side light emitting component 12b: Bottom light emitting component
120a: Red LED 120b: Blue LED
120c: RGB LED 120d: UV-B LED

1. A therapy mouth guard comprising:
a guard body configured to accommodate teeth and gums by having a curvature corresponding to the contour accommodating occlusal surfaces of teeth facing the occlusal surfaces and buccal surfaces facing teeth and gums from inner and outer sides; and
at least one side illumination component installed on at least one of inner and outer side wall parts of the guard body to illuminate light toward lateral surfaces of gums and teeth, wherein the side illumination component is composed of multiple layers to overlap light for increasing illumination intensity.

2. The therapy mouth guard of claim 1, wherein the side emission component comprises:
a first emission component array composed of a plurality of first light-emitting components configured to emit light of a first wavelength for irradiation; and a second emission component array composed of a plurality of second light-emitting components configured to emit light of a second wavelength for irradiation, wherein the first and second emission component arrays are arranged alternately.

3. The therapy mouth guard of claim 2, wherein the first light-emitting component array and the second light-emitting component array are arranged alternately.

4. The therapy mouth guard of claim 3, wherein the first light-emitting component array and the second light-emitting component array form horizontal rows along the longitudinal direction of the guard body.

5. The therapy mouth guard of claim 4, wherein the horizontal rows of the first light-emitting component and the second light-emitting component alternate from the gum towards the teeth, such that light emitted from the first wavelength by the horizontal rows corresponding to the first column of the first light-emitting component and the third column of the first light-emitting component overlaps and illuminates the gum, and light emitted from the second wavelength by the horizontal rows corresponding to the second column of the second light-emitting component and the fourth column of the second light-emitting component overlaps and illuminates the gingival margin.

6. The therapy mouth guard of claim 4, wherein the horizontal rows of the first light-emitting component and the horizontal rows of the first light-emitting component are configured to draw a waveform corresponding to the gingival margin.

7. The therapy mouth guard of claim 3, wherein the first light-emitting component and the second light-emitting component form vertical columns along the vertical direction of the guard body.

8. The therapy mouth guard of claim 7, wherein light of the first wavelength generated from vertical columns of the first light-emitting components neighboring each other on both sides of the vertical columns of the second light-emitting components is overlapped and irradiated onto the gums and teeth, and light of the second wavelength generated from vertical columns of the second light-emitting components neighboring each other on both sides of the vertical columns of the first light-emitting components is overlapped and irradiated onto the gums and teeth.

9. The therapy mouth guard of claim 3, wherein the first light-emitting components and the second light-emitting components form diagonal rows with respect to the gingival boundary surface on the guard body.

10. The therapy mouth guard of claim 9, wherein the second light-emitting components form diagonal rows with respect to the gingival boundary surface, and light of the first wavelength generated from diagonal rows of the first light-emitting components, which are adjacent on both sides of the diagonal rows of the second light-emitting components, overlaps and is irradiated onto the gingiva and teeth, and light of the second wavelength generated from diagonal rows of the second light-emitting components, which are adjacent on both sides of the diagonal rows of the first light-emitting components, overlaps and is irradiated onto the gingiva and teeth.

11. The therapy mouth guard of claim 1, wherein the first light-emitting components and the second light-emitting components are each equipped with RGB light-emitting components capable of selectively emitting light of one wavelength from among a plurality of wavelengths.

12. The therapy mouth guard of claim 11, wherein the RGB light-emitting components form horizontal rows along the length direction of the guard body, and wherein the horizontal rows emitting light of the first wavelength and the horizontal rows emitting light of the second wavelength are arranged in a repeating pattern with a certain pattern.

13. The therapy mouth guard of claim 12, wherein the RGB light-emitting components cause the light of the first wavelength to overlap with the gums from the horizontal rows of the RGB light-emitting components corresponding to the first column and the third column, and wherein the light of the second wavelength emitted from the horizontal rows of the RGB light-emitting components corresponding to the second column and the fourth column overlaps with the gingival boundary surface for inspection.

14. The therapy mouth guard of claim 11, wherein the RGB light-emitting components form vertical rows along the vertical direction of the guard body from the gums to the teeth, and wherein the vertical rows emitting light of the first wavelength and the vertical rows emitting light of the second wavelength are arranged in a regular pattern alternating repeatedly.

15. The therapy mouth guard of any one of claims 2 to 14, wherein the light of the first wavelength is red light and the light of the second wavelength is blue light.

16. The therapy mouth guard of claim 1, wherein at least some of the first light emitting elements and the second light emitting elements generate far-UVC (far-ultraviolet) radiation for irradiation.

17. The therapy mouth guard of claim 1, wherein at least some of the first light emitting elements and the second light emitting elements generate ultraviolet-B (UV-B) radiation for irradiation.

18. The therapy mouth guard of claim 1, further comprising a buccal surface light emitting element installed on the buccal surface facing the buccal surface of the tooth on the buccal surface of the guard body, capable of irradiating light toward the buccal surface of the tooth.

19. The therapy mouth guard of claim 18, wherein the buccal surface light emitting element is equipped with a blue light emitting element capable of irradiating blue light for the purpose of sterilizing the teeth.

20. The therapy mouth guard of claim 19, wherein the blue light emitting element forms multiple horizontal rows along the length direction of the guard body, and the blue light emitted from the blue light emitting element is irradiated onto the occlusal surface of the teeth in an overlapping manner, thereby increasing the irradiation intensity of the blue light.

21. The therapy mouth guard of claim 18, wherein the occlusal surface light emitting element is equipped with an RGB light emitting element capable of selectively emitting light of any one wavelength among multiple wavelength bands.

22. The therapy mouth guard of claim 18, wherein at least a portion of the occlusal surface light emitting elements emit far-UVC radiation for irradiation.

23. The therapy mouth guard of claim 18, wherein at least a portion of the occlusal surface light emitting elements emit ultraviolet-B radiation for irradiation.

24. The light therapy mouth guard of claim 1, wherein a plurality of ultrasonic vibrators arranged along the length direction of the mouth guard body are embedded in the mouth guard body to induce cleaning of teeth by applying ultrasonic vibration to the teeth.

25. The light therapy mouth guard of claim 24, further comprising a vibration transmitting member configured such that when a protruding portion of the vibration transmitting member contacts the teeth while being connected to the ultrasonic vibrator, which is embedded in the mouth guard body, ultrasonic vibrations of the ultrasonic vibrator are applied to the teeth.

26. In claim 24, the ultrasonic vibrators are arranged along the length direction of the mouth guard body to be capable of individually addressing each tooth, allowing for independent operation. This configuration enables the application of ultrasonic vibrations to each tooth with time gaps according to a program or allows for concentrated ultrasonic vibration on specific teeth.

27. In claim 27, two mouth guards are configured as a pair to be worn together on the upper and lower jaws. Each mouth guard in the pair worn on the upper and lower jaws is equipped with side illumination elements, which are integratedly controllable via a communication module, allowing for synchronized control.

28. In claim 28, the mouth guards are configured as a pair to be worn together on the upper and lower jaws. Each mouth guard in the pair is equipped with fastening means, such as a connecting member, to securely interlock with each other when worn on the upper and lower jaws simultaneously, facilitating their coordinated use.

29. In claim 29, the receptacles of the guard body consist of an upper receptacle, open towards the upper side, and a lower receptacle, open towards the lower side, enabling simultaneous wearing on the upper and lower jaws. Each of these receptacles houses the lateral illumination elements.

30. The mouth guard for light therapy according to claim 1, further comprising a controller for controlling the operation of the side light emitting device and a communication module for communication with the outside.

31. Mouth guard for light therapy of claim 20; A terminal installed with a dedicated app capable of controlling the side light emitting device through the controller; A light therapy system comprising:

32. The method of claim 31, wherein the first light-emitting device and the second light-emitting device are each provided as an RGB light-emitting device capable of selectively generating light in one of a plurality of wavelength bands, and the dedicated app includes one of a plurality of colors. A color palette is provided so that the user can select one color, and when the user selects a color from the color palette of the dedicated app, the controller controls the RGB light emitting device to emit light of the corresponding color. A light therapy system that works.

33. The method of claim 32, wherein a tooth row image is displayed on the display of the terminal on which the dedicated app is executed, and only some of the side light emitting elements emit light by clicking on individual tooth images belonging to the tooth row image. A light therapy system characterized by selection.

34. The method of claim 31, further comprising a chewing surface light-emitting device installed on the chewing surface facing portion of the guard body to irradiate light toward the chewing surface of the teeth, and the dedicated app is operated on the chewing surface through the controller. A light therapy system, characterized in that the light-emitting elements are controllable and only some light-emitting elements of the side light-emitting elements and the chewing surface light-emitting elements are selected to emit light by clicking on individual tooth images on the display of the terminal.

35. The method of claim 34, wherein the bottom light emitting device is provided as an RGB light emitting device capable of selectively generating light in one of a plurality of wavelength bands, and the dedicated app allows the user to select one of a plurality of colors. A light therapy system characterized in that a color palette is prepared so that when the user selects a color from the color palette of the dedicated app, the controller controls the RGB light emitting device to emit light of the corresponding color.
